(19) United States
(12) Patent Application Publication
(10) Pub. No.: US 2021/0212609 A1
(43) Pub. Date: Jul. 15, 2021

(54) Component Concentration Measurement Device

(71) Applicant: Nippon Telegraph and Telephone Corporation, Tokyo (JP)

(72) Inventors: Masahito Nakamura, Tokyo (JP); Yujiro Tanaka, Tokyo (JP); Michiko Seyama, Tokyo (JP); Katsuhiro Ajito, Tokyo (JP); Daichi Matsunaga, Tokyo (JP)

(21) Appl. No.: 17/253,422

(22) PCT Filed: May 24, 2019
(86) PCT No.: PCT/JP2019/020664

§ 371 Date: Dec. 17, 2020

(30) Foreign Application Priority Data
Jun. 21, 2018 (JP) 2018-117636

(51) Int. Cl. A61B 5/145; A61B 5/00; A61B 5/107

(52) U.S. Cl. CPC A61B 5/14532; A61B 5/1075; A61B 5/0095

(57) ABSTRACT

A component concentration measurement device includes a light application unit that applies beam light of a wavelength that is absorbed by glucose to a site of measurement, and a detection unit that detects a photoacoustic signal which is generated at the site of measurement where the beam light emitted from the light application unit has been applied. The component concentration measurement device also includes a thickness measurement unit that measures a thickness of the site of measurement, and a correction unit that corrects an acoustic signal detected by the detection unit with the thickness measured by the thickness measurement unit.

### CROSS-REFERENCE TO RELATED APPLICATIONS

This application is a national phase entry of PCT Application No. PCT/JP2019/020664, filed on May 24, 2019, which claims priority to Japanese Application No. 2018-117636, filed on Jun. 21, 2018, which applications are hereby incorporated herein by reference.

### TECHNICAL FIELD

The present invention relates to a component concentration measurement device for non-invasively measuring glucose concentration.

### BACKGROUND

In terms of determining a dose of insulin for a diabetes patient or preventing diabetes, it is important to know (measure) blood sugar level. The blood sugar level is the concentration of glucose in blood, and as a way of measuring this kind of component concentration, a photoacoustic method is well known (see Patent Literature 1).

When a certain amount of light (an electromagnetic wave) is applied to a living body, the applied light is absorbed by molecules contained in the living body. As a result, target molecules for measurement in a portion applied with the light are locally heated to expand and generate a sound wave. The pressure of the sound wave depends on the amount of molecules that absorb the light. The photoacoustic method measures this sound wave to measure the amount of molecules in the living body. A sound wave is a pressure wave that propagates within a living body and has a property of being resistant to scattering compared to an electromagnetic wave; the photoacoustic method can be regarded to be a suitable way for measuring blood components in a living body.

Measurement by the photoacoustic method enables continuous monitoring of the glucose concentration in blood. In addition, measurement with the photoacoustic method does not require blood sample and causes no discomfort in a subject of measurement.

### CITATION LIST

### Patent Literature

Patent Literature 1: Japanese Patent Laid-Open No. 2010-104858.

### SUMMARY

### Technical Problem

A site on a human body that is subjected to this type of measurement changes in thickness over time. For example, in this kind of measurement, a detection unit is attached to a lobulus auriculae (ear lobe). The lobulus auriculae, however, is an easily deforming part of a human body such that its thickness changes when the detection unit is attached for a long time. When the thickness at the site of measurement thus changes, however, a measurement result of glucose measurement in a human body by the photoacoustic method will change. As the measurement result changes due to such a change in the thickness of the site of measurement, it can happen that concentrations are actually the same when results that were measured at different times are different or that concentrations are actually different when results that were measured at different times are the same, which hinders an accurate measurement.

In order to solve such a drawback, an object of embodiments of the present invention is to suppress decrease in measurement accuracy that is caused by a change of a human body over time when glucose in a human body is measured by the photoacoustic method.

### Means for Solving the Problem

A component concentration measurement device according to embodiments of the present invention includes: a light application unit that applies beam light of a wavelength that is absorbed by glucose to a site of measurement; a detection unit that detects a photoacoustic signal which is generated at the site of measurement where the beam light emitted from the light application unit has been applied; a thickness measurement unit that measures a thickness of the site of measurement; and a correction unit that corrects an acoustic signal detected by the detection unit with the thickness measured by the thickness measurement unit.

In the component concentration measurement device, the light application unit and the detection unit are positioned opposite each other across the site of measurement, and the thickness measurement unit measures the thickness of the site of measurement between the light application unit and the detection unit.

In the component concentration measurement device, the thickness measurement unit determines the thickness of the site of measurement by optical coherence tomography of the site of measurement.

In the component concentration measurement device, the thickness measurement unit determines the thickness of the site of measurement by ultrasonic tomography of the site of measurement.

In the component concentration measurement device, the light application unit may include a light source unit that generates the beam light of a wavelength that is absorbed by glucose; and a pulse control unit that turns the beam light generated by the light source unit into pulsed light of a set pulse width.

### Effects of Embodiments of the Invention

As has been described above, according to embodiments of the present invention, the thickness of the site of measurement is measured and an acoustic signal detected by the detection unit is corrected with the measured thickness. Thus, it provides an advantageous effect of suppressing decrease in the measurement accuracy that is caused by change in a human body over time when glucose in the human body is measured by the photoacoustic method.

### BRIEF DESCRIPTION OF THE DRAWINGS

FIG. 1 is configuration diagram showing a configuration of a component concentration measurement device in an embodiment of the present invention.

FIG. 2 is a configuration diagram showing a more detailed configuration of a light source unit 105 and a detection unit 102 in an embodiment of the present invention.

FIG. 3 is a configuration diagram showing a more detailed configuration of a thickness measurement unit 103 in an embodiment of the present invention.

FIG. 4 is a characteristic diagram showing an experiment result for a measurement of glucose concentration in a living body with the component concentration measurement device in an embodiment.

### DETAILED DESCRIPTION OF ILLUSTRATIVE EMBODIMENTS

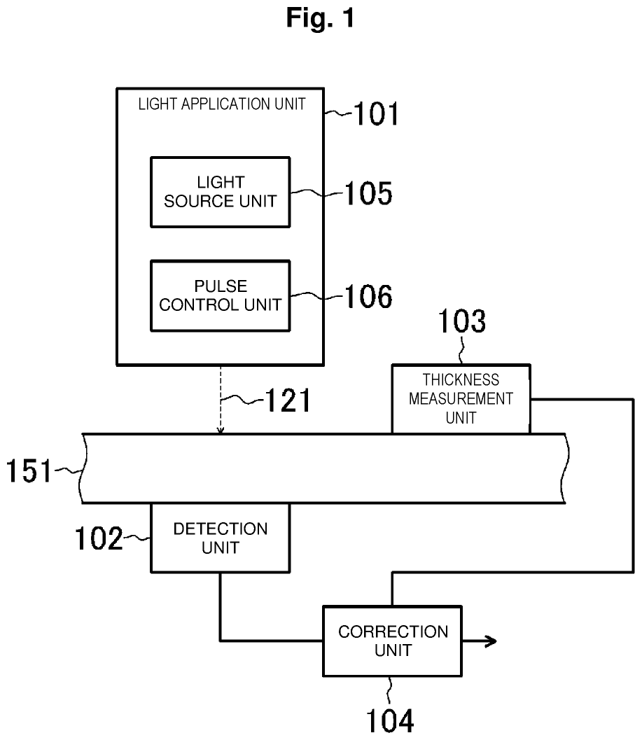

A component concentration measurement device according to an embodiment of the present invention is described below with reference to FIG. 1. The component concentration measurement device includes a light application unit 101 that applies beam light of a wavelength that is absorbed by glucose to a site of measurement 151, and a detection unit 102 that detects a photoacoustic signal generated in the site of measurement 151 where the beam light emitted from the light application unit 101 has been applied.

For example, the light application unit 101 includes a light source unit 105 that generates the beam light 121 of a wavelength that is absorbed by glucose, and a pulse control unit 106 that turns the beam light 121 generated by the light source into pulsed light of a set pulse width. Glucose exhibits absorbency in light wavelength bands around 1.6 μm and around 2.1 lam (see Patent Literature 1). The beam light 121 has a beam diameter of about 100 μm, for example. Although not shown, shaping of the beam light may be performed using a lens or a collimator, such as turning the beam light 121 into collimated light.

The component concentration measurement device also includes a thickness measurement unit 103 that measures the thickness of the site of measurement 151, and a correction unit 104 that corrects an acoustic signal detected by the detection unit 102 with the thickness measured by the thickness measurement unit 103. Here, the light application unit 101 and the detection unit 102 are positioned opposite each other across the site of measurement 151. The thickness measurement unit 103 substantially measures the thickness of the site of measurement 151 in an area between the light application unit 101 and the detection unit 102. The thickness measurement unit 103 can be positioned near the locations where the light application unit 101 and the detection unit 102 are positioned.

The thickness measurement unit 103 determines the thickness of site of measurement 151 by optical coherence tomography of the site of measurement 151, for example. Alternatively, the thickness measurement unit 103 determines the thickness of site of measurement 151 by ultrasonic tomography of the site of measurement 151. The site of measurement 151 is a portion of a human body, like an ear lobe, for example.

The correction unit 104 corrects the acoustic signal detected by the detection unit 102 with the thickness measured by the thickness measurement unit 103 within a preset time after the point when the detection unit 102 detected the acoustic signal. For example, the correction unit 104 corrects the acoustic signal detected by the detection unit 102 with the thickness measured by the thickness measurement unit 103 at the point the detection unit 102 has detected the acoustic signal.

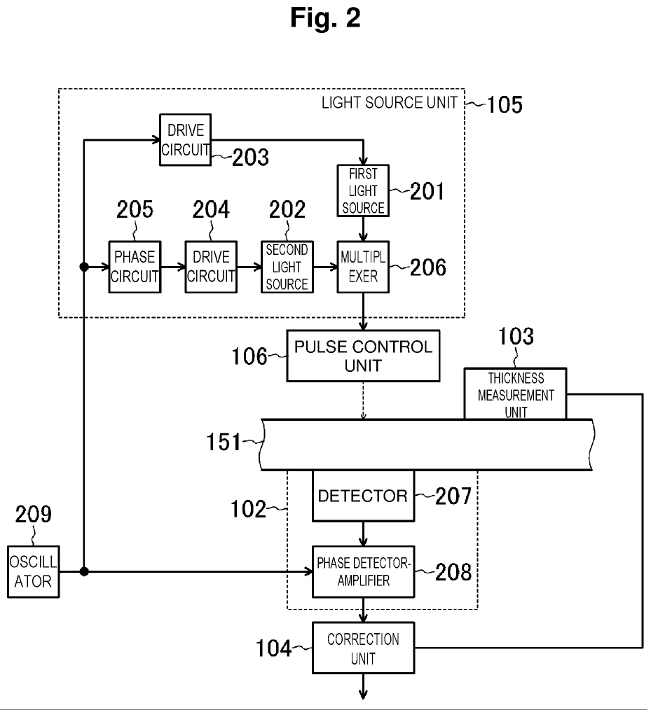

The light source unit 105 includes a first light source 201, a second light source 202, a drive circuit 203, a drive circuit 204, a phase circuit 205, a multiplexer 206, a detector 207, a phase detector-amplifier 208, and an oscillator 209 as shown in FIG. 2. The first light source 201, the second light source 202, the drive circuit 203, the drive circuit 204, the phase circuit 205, and the multiplexer 206 constitute the light source unit 105. The detector 207 and the phase detector-amplifier 208 constitute the detection unit 102.

The oscillator 209 is connected to each of the drive circuit 203, the phase circuit 205, and the phase detector-amplifier 208 via signal wires. The oscillator 209 sends a signal to each of the drive circuit 203, the phase circuit 205, and the phase detector-amplifier 208.

The drive circuit 203 receives the signal sent from the oscillator 209, and supplies driving electric power to the first light source 201, which is connected by a signal wire, to cause the first light source 201 to emit light. The first light source 201 is a semiconductor laser, for example.

The phase circuit 205 receives the signal sent from the oscillator 209, and sends a signal generated by giving a phase shift of 180° to the received signal to the drive circuit 204, which is connected by a signal wire.

The drive circuit 204 receives the signal sent from the phase circuit 205, and supplies driving electric power to the second light source 202, which is connected by a signal wire, to cause the second light source 202 to emit light. The second light source 202 is a semiconductor laser, for example.

The first light source 201 and the second light source 202 output light of different wavelengths from each other and direct their respective output light to the multiplexer 206 via light wave transmission means. For the first light source 201 and the second light source 202, the wavelength of light of one of them is set to a wavelength that is absorbed by glucose, while the wavelength of light of the other is set to a wavelength that is absorbed by water. Their respective wavelengths are also set such that degrees of their absorption will be equivalent.

The light output by the first light source 201 and the light output by the second light source 202 are multiplexed in the multiplexer 206 and are incident onto the pulse control unit 106 as one light beam. Upon incidence of the light beam, in the pulse control unit 106, the incident light beam is applied to the site of measurement 151 as pulsed light of a predetermined pulse width. Inside the site of measurement 151 thus applied with the pulsed light beam, a photoacoustic signal is generated.

The detector 207 detects the photoacoustic signal generated in the site of measurement 151, converts it into an electric signal, and sends it to the phase detector-amplifier 208, which is connected by a signal wire. The phase detector-amplifier 208 receives a synchronization signal necessary for synchronous detection sent from the oscillator 209, and also receives the electric signal proportional to the photoacoustic signal being sent from the detector 207, performs synchronous detection, amplification and filtering on it, and outputs an electric signal proportional to the photoacoustic signal.

The first light source 201 outputs light that has been intensity-modulated in synchronization with an oscillation frequency of the oscillator 209. In contrast, the second light source 202 outputs light that has been intensity-modulated with the oscillation frequency of the oscillator 209 and in synchronization with the signal that has gone through a phase shift of 180° in the phase circuit 205.

Here, since the intensity of the signal output by the phase detector-amplifier 208 is proportional to the amount by which the light output from each of the first light source 201 and the second light source 202 was absorbed by components (glucose, water) in the site of measurement 151, the intensity of the signal is proportional to the amounts of components in the site of measurement 151.

As mentioned above, the light output by the first light source 201 and the light output by the second light source 202 have been intensity-modulated with signals of the same frequency. Accordingly, there is no effect of unevenness in frequency characteristics of a measurement system, which is problematic in the case of intensity modulation with signals of multiple frequencies.

Meanwhile, non-linear dependence on absorption coefficient that exists in measured values of photoacoustic signals, which is problematic in measurements by the photoacoustic method, can be solved by performing measurements using light of multiple wavelengths that gives an equal absorption coefficient as described above (see Patent Literature 1).

As mentioned above, the strength of the acoustic signal output from the detection unit 102 is corrected by the correction unit 104, and based on a corrected correction value, a component concentration derivation unit (not shown) determines the amount of glucose component in blood within the site of measurement 151.

Next, correction performed in the correction unit 104 for the acoustic signal detected by the detection unit 102 with the thickness of the site of measurement 151 measured by the thickness measurement unit 103 is described.

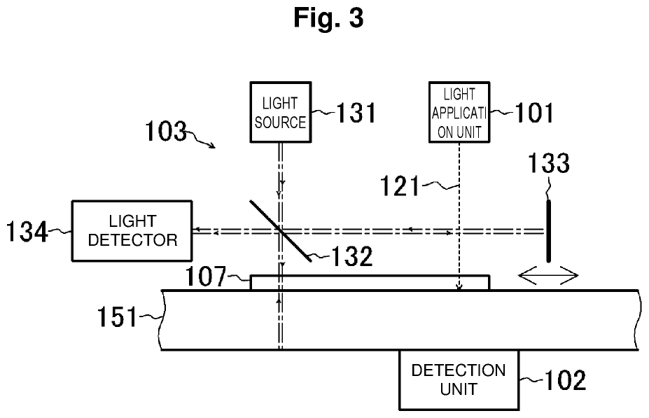

First, the thickness measurement unit 103 is described in more detail. The thickness measurement unit 103 is a known optical coherence tomography (OCT) device including a light source 131, a beam splitter 132, a mirror 133, and a light detector 134, as shown in FIG. 3, for example. In this example, it includes a collimator 107 for turning the beam light 121 into collimated light.

Light emitted by the light source 131 branches into two in the beam splitter 132, one of which is to be incident on the site of measurement 151 via the collimator 107 and the other is to be incident on the mirror 133. The light incident on one side of the site of measurement 151 is reflected at the other side of the site of measurement 151, where there is a difference in refractive index between internal tissue inside the site of measurement 151 and the outside of the site of measurement 151, and again exits from the one side of the site of measurement 151.

The light that has thus returned from the site of measurement 151 and the light reflected on the mirror 133 are superposed in the beam splitter 132. At this point, due to interference of light, the two lights strengthen one another if the distances they have traveled are equal, whereas they cancel one another out if there is a disparity in their distances. By moving the mirror 133 and determining a position where the two lights interfere with and strengthen one another via detection of light intensity with the light detector 134, the distances traveled by the lights through the site of measurement 151 can be known and the thickness of the site of measurement 151 can be known.

Next, a photoacoustic signal detected by the detection unit 102 is described. In a one-dimensional system, a photoacoustic signal at a time t for a substance having a certain concentration distribution is represented as Formula (1).

$$\begin{matrix}Formula 1 & \\ P ( t ) = {\int }_{0}^{∞} β ( x , t ) exp ( - \frac{( 1 + j ) x}{{μ}_{s}} ) dx & ( 1 )\end{matrix}$$

In Formula (1), P is the output of the photoacoustic signal, β(x) is an absorption coefficient at depth x and at a given wavelength when a radiation end surface of the light source is defined as x=0, and μₛ is thermal diffusion length. A generated sound wave (photoacoustic signal) causes a resonance phenomenon between the collimator 107 and the detection unit 102, which enables an amplified acoustic signal to be acquired.

Here, a qth-order resonant mode of an acoustic signal is represented by Formula (2) below.

$$\begin{matrix}Formula 2 & \\ q = \frac{2 fL}{v} & ( 2 )\end{matrix}$$

In Formula (2), ν is the speed of sound, f is a modulation frequency of light, and L is the thickness of the site of measurement 151.

If the thickness of the site of measurement 151 changes due to change over time, the measurement accuracy will decrease due to simultaneous changes in the resonant mode of the sound wave and the component being measured. Accordingly, the thickness of the site of measurement 151 is measured by performing an OCT measurement. The mirror 133 is driven about 5 to 8 mm and thickness L(t) at a time t is measured. Measurement start time is defined as to.

The resonant mode is represented by Formula (3) below.

$$\begin{matrix}Formula 3 & \\ q = \frac{2 fL ( t )}{v} = \frac{2 f Δ L \cdot L ( t 0 )}{v} & ( 3 )\end{matrix}$$

Here, “ΔL=L(t)/L(t0) (4)”.

With adjustment of the modulation frequency such that “f′=f/ΔL . . . (5)”, q in Formula (3) can assume the same resonant mode as an initial value. By setting such a modulation frequency that maximizes sensitivity at time to, photoacoustic measurements can be performed in a resonant mode with high sensitivity at all times even if there is a change in the state of the site of measurement 151 due to temporal change.

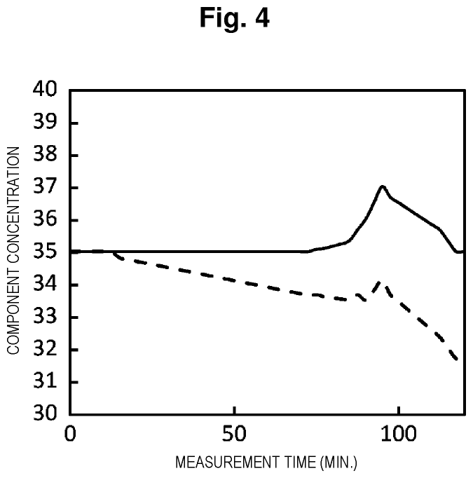

FIG. 4 shows an experiment result for a measurement of glucose concentration in a living body with the component concentration measurement device according to the above-described embodiment. In FIG. 4, the broken line indicates before correction and the solid line indicates after correction. As shown in FIG. 4, according to the embodiment, the effect of moisture content is suppressed, which enables an accurate measurement of the target component concentration.

As has been described above, according to embodiments of the present invention, the thickness of the site of measurement is measured and an acoustic signal detected by the detection unit is corrected with the measured thickness. Thus, it is possible to suppress decrease in the measurement accuracy that is caused by a change in a human body over time when glucose in the human body is measured by the photoacoustic method.

It will be apparent that the present invention is not limited to the above-described embodiments but many variations and combinations may be made by ordinarily skilled persons in the art within the technical idea of the invention.

### REFERENCE SIGNS LIST

101 light application unit
102 detection unit
103 thickness measurement unit
104 correction unit
105 light source unit
106 pulse control unit
121 beam light
151 site of measurement.

1.-5. (canceled)

6. A component concentration measurement device comprising:
a light applicator configured to apply beam light of a wavelength that is absorbed by glucose to a site of measurement;
a detector configured to detect a photoacoustic signal generated at the site of measurement where the beam light has been applied;
a thickness measurer configured to measure a thickness of the site of measurement; and
a corrector configured to correct an acoustic signal detected by the detector with the thickness measured by the thickness measurer.

7. The component concentration measurement device according to claim 6, wherein:
a light applicator and the detector are positioned opposite each other across the site of measurement, and
the thickness measurer is configured to measure the thickness of the site of measurement between the light applicator and the detector.

8. The component concentration measurement device according to claim 6, wherein the thickness measurer is configured to determine the thickness of the site of measurement by optical coherence tomography of the site of measurement.

9. The component concentration measurement device according to claim 6, wherein the thickness measurer is configured to determine the thickness of the site of measurement by ultrasonic tomography of the site of measurement.

10. The component concentration measurement device according to claim 6, wherein the light applicator comprises:
a light source that generates the beam light of a wavelength that is absorbed by glucose; and
a pulse controller that turns the beam light generated by the light source into pulsed light of a set pulse width.

11. A method comprising:
applying, by a light applicator, beam light of a wavelength that is absorbed by glucose to a site of measurement;
detecting, by a detector, a photoacoustic signal generated at the site of measurement where the beam light has been applied;
measuring a thickness of the site of measurement; and
correcting an acoustic signal generated at the site of measurement with the thickness of the site of measurement.

12. The method of claim 11, wherein:
a light applicator and the detector are positioned opposite each other across the site of measurement, and
measuring the thickness comprises measuring the thickness of the site of measurement between the light applicator and the detector.

13. The method of claim 11, wherein measuring the thickness comprises determining the thickness of the site of measurement by optical coherence tomography of the site of measurement.

14. The method of claim 11, wherein measuring the thickness comprises determining the thickness of the site of measurement by ultrasonic tomography of the site of measurement.

15. The method of claim 11, wherein the light applicator comprises:
a light source that generates the beam light of a wavelength that is absorbed by glucose; and
a pulse controller that turns the beam light generated by the light source into pulsed light of a set pulse width.
